(19) United States
(12) Patent Application Publication
(10) Pub. No.: US 2019/0224214 A1
(43) Pub. Date: Jul. 25, 2019

(54) HUMAN GHRELIN O-ACYLTRANSFERASE INHIBITORS

(71) Applicants: James L. Hougland, Fayetteville, NY (US); John D. Chisholm, Fayetteville, NY (US)

(72) Inventors: James L. Hougland, Fayetteville, NY (US); John D. Chisholm, Fayetteville, NY (US)

(73) Assignee: SYRACUSE UNIVERSITY, SYRACUSE, NY (US)

(21) Appl. No.: 16/324,985

(22) PCT Filed: Aug. 15, 2017
(86) PCT No.: PCT/US17/46863

§ 371 Date: Feb. 12, 2019

Related U.S. Application Data
(60) Provisional application No. 62/375,699, filed on Aug. 16, 2016.

(51) Int. Cl. A61K 31/566; A61K 31/122; A61P 3/04

(52) U.S. Cl. CPC A61K 31/566; A61P 3/04; A61K 31/122

(57) ABSTRACT

A class of cyanosteroid compounds that efficiently inhibit ghrelin acylation by ghrelin O-acyltransferase. The compounds have a steroid scaffold with α,β-unsaturated ketone in the A ring position such an α-cyanoenone. Exemplary compounds include (5S,8S,9S,10S,13S,14S)-10,13-dimethyl-3-oxo-4,5,6,7,8,9,10,11,12,13,14,15,16,17-tetradecahydro-3H-cyclopenta[a]phenanthrene-2-carbonitrile.

### CROSS-REFERENCE TO RELATED APPLICATIONS

The present application claims priority to U.S. Provisional Application No. 62/375,699 filed on Aug. 16, 2016.

### BACKGROUND OF THE INVENTION

### 1. Field of the Invention

The present invention relates to ghrelin O-acyltransferase inhibitors and, more particularly, to a class of cyanosteroid compounds that efficiently inhibit ghrelin acylation by ghrelin O-acyltransferase.

### 2. Description of the Related Art

The increasing incidence of diabetes and obesity within the American population presents an urgent and growing threat to public health. The prevalence of type II diabetes has risen dramatically, with ˜26 million adults and children suffering from diabetes and an estimated 79 million American adults classified as pre-diabetic as of 2011. The global picture is also foreboding, with 382 million people suffering from diabetes in 2013, a figure expected to increase to 592 million by 2035. These sobering statistics underscore the need for new therapeutic avenues to treat these diseases, particularly small-molecule based treatments to complement the lifestyle modification and surgical approaches now in use.

The peptide hormone ghrelin presents a promising and unexploited target for development of small-molecule therapeutics to treat obesity, diabetes, and a range of other health conditions. Ghrelin is a 28-amino acid secreted peptide, discovered in 1999, which has been implicated in a wide array of physiological processes. Ghrelin is perhaps most well-known for its ability to stimulate appetite, but ghrelin has been linked to maintenance of body energy balance through regulation of fat mass and modulation of insulin signaling and glucose metabolism sensitivity, while des-acyl ghrelin and des-acyl ghrelin analogs block some of these effects. Elevated ghrelin levels in neonatal mice have been linked to metabolic dysregulation later in life, suggesting the potential for inhibition of ghrelin signaling in infants for prophylactic treatment of obesity and metabolic disturbances in the presence of elevated ghrelin levels as in Prader-Willi syndrome.

Ghrelin-dependent pathways present attractive targets for drug development, as ghrelin requires multiple covalent modifications for biological activity. Ghrelin maturation involves a unique posttranslational modification of the third serine from the N-terminus of the 94-amino acid ghrelin precursor des-acyl proghrelin, wherein this serine is acylated by an octanoyl (C8) fatty acid group (FIG. 1). Ghrelin O-acyltransferase (GOAT), the integral membrane enzyme shown to be responsible for acylation of both des-acyl proghrelin and the unacylated form of mature 28-amino acid ghrelin, was identified in 2008. While both acylated ghrelin and des-acyl ghrelin are present in blood serum, only the acylated form of ghrelin (hereafter referred to as “ghrelin”) can bind and activate signaling through its cognate GHSR-1a receptor. Ghrelin is the only substrate predicted for GOAT within the human proteome, which reduces the potential for off-target effects due to the loss of GOAT-catalyzed acylation of other protein targets upon inhibition of GOAT activity. The unique and essential nature of ghrelin octanoylation makes this modification an ideal target for inhibiting ghrelin activity.

While the potential of ghrelin signaling as a therapeutic target has been discussed in the literature for several years, the lack of potent inhibitors targeting this pathway has hampered efforts towards evaluating this approach. Some examples of GOAT inhibitors based on either mimics of ghrelin/acylated ghrelin or screening of small molecule libraries have been reported (FIG. 1b). The strongest evidence supporting the potential for GOAT inhibitors to modulate serum levels of acylated ghrelin comes a peptide-based bisubstrate mimetic GO-CoA-Tat inhibitor. This inhibitor effectively inhibited GOAT in both cultured mammalian cells and mice, and treatment of mice with this inhibitor increased glucose tolerance and reduced weight gain. However, the pharmaceutical utility of GO-CoA-Tat is limited by its susceptibility to proteolytic degradation and its large size (MW ˜3600 Da). Although a limited number of small molecule GOAT inhibitors have been described, none of these compounds have been reported to exhibit inhibition of ghrelin octanoylation in cell- or animal-based studies. The absence of readily available small molecule GOAT inhibitors remains the principal obstacle in validating ghrelin and ghrelin-related signaling pathways as treatment avenues. As a result, there is a need in the art for small molecules with inhibitory activity against GOAT to address the challenge in exploiting ghrelin signaling for therapeutic development and catalyze the creation of potent “drug-like” inhibitors of GOAT.

### BRIEF SUMMARY OF THE INVENTION

The present invention provides a class of cyanosteroid compounds that efficiently inhibit ghrelin acylation by ghrelin O-acyltransferase and thus addresses the challenge in exploiting ghrelin signaling for therapeutic development and the creation of potent “drug-like” inhibitors of GOAT. The compounds have a steroid scaffold with α,β-unsaturated ketone in the A ring position such an α-cyanoenone. Exemplary compounds include (5S,8S,9S,10S,13S,14S)-10,13-dimethyl-3-oxo-4,5,6,7,8,9,10,11,12,13,14,15,16,17-tetradecahydro-3H-cyclopenta[a]phenanthrene-2-carbonitrile. The present invention also comprises a method of inhibiting human ghrelin O-acyltransferase by administering a compound comprising a steroid scaffold having α,β-unsaturated ketone in an A ring position. The α,β-unsaturated ketone may comprise an α-cyanoenone.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1A is a schematic of ghrelin O-acyltransferase (GOAT) as a target for blocking ghrelin signaling showing ghrelin octanoylation catalyzed by GOAT;

FIG. 1B is a schematic of the structures of reported GOAT inhibitors where Ahx denotes aminohexanoate and Tat denotes a Tat peptide sequence;

FIG. 2A is a schematic of how an hGOAT inhibition profile supports the involvement of a catalytically essential cysteine residue using a topological model of hGOAT, with cysteines residues highlighted

FIG. 2B is a graph of the inhibition of hGOAT octanoylation activity by N-ethylmaleimide (NEM, structure shown in inset);

FIG. 3C is a graph of inhibitor dilution assays reveal irreversible hGOAT inhibition by NEM and reversible inhibition by CDDO-EA (3) and α-cyanoenone steroid 9 where Dap-C8 denotes the GS(octanamide-Dap)FL product-mimetic GOAT inhibitor used as a control for reversible inhibition, reactions were performed and analyzed to determine percent activity, IC₅₀ values, and inhibitor reversibility, and errors bars reflect the standard deviation from a minimum of three determinations;

FIG. 3A is a graph of the inhibition of hGOAT octanoylation activity by compound 9.

FIG. 3B is a graph of the inhibition of hGOAT octanoylation activity by compound 10.

FIG. 3C is a graph of the inhibition of hGOAT octanoylation activity by compound 11;

FIG. 4 is schematic of a class of hGOAT inhibitors according to the present invention; and

FIG. 5 is a schematic of a synthesis of a class of hGOAT inhibitors according to the present invention.

### DETAILED DESCRIPTION OF THE INVENTION

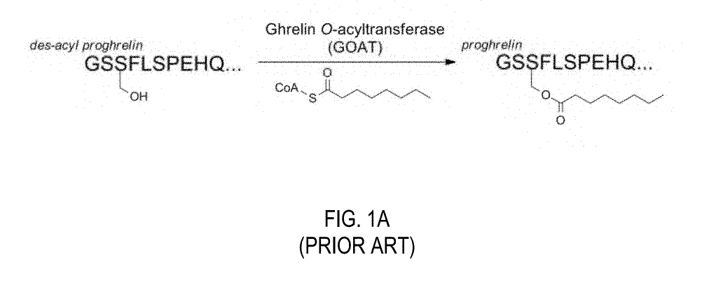

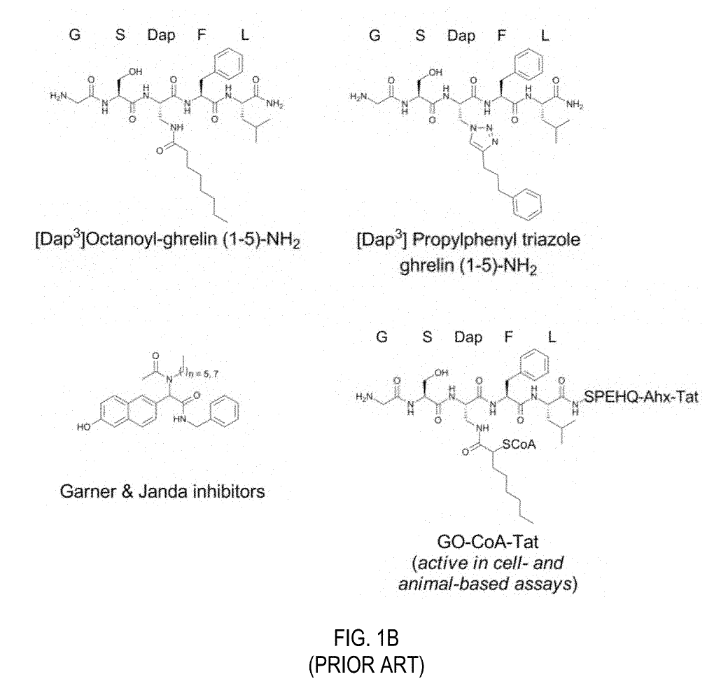

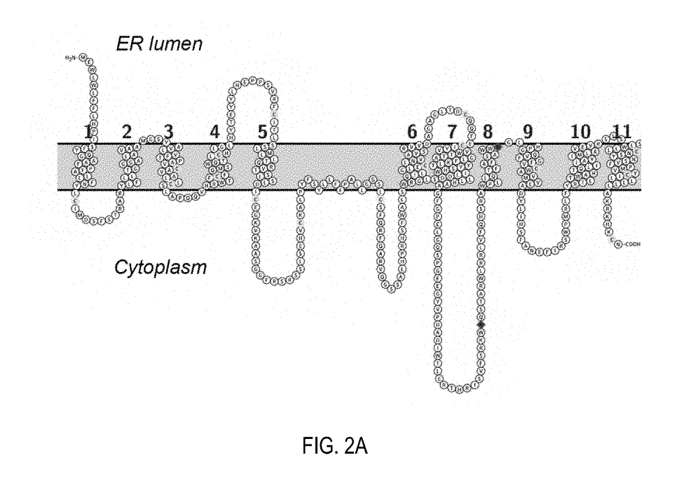

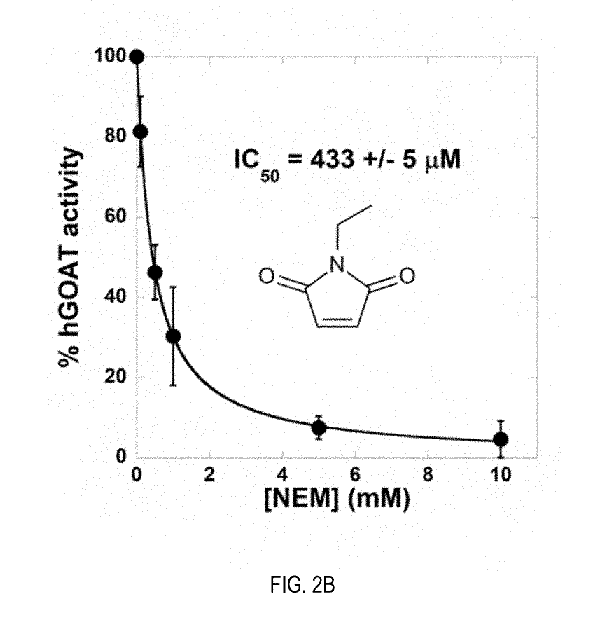

Referring to the figures, wherein like numerals refer to like parts throughout, there is seen in FIGS. 1 through 5 a class of cyanosteroid compounds that efficiently inhibit ghrelin acylation by ghrelin O-acyltransferase.

### Example

Results

Library Screening Reveals a New Small Molecule GOAT Inhibitor

The majority of reported GOAT inhibitors are substrate- or product-mimetic compounds. While effective to varying degrees in in vitro enzyme assays, many of these compounds appear to lack sufficient cell permeability to permit effective inhibition of ghrelin octanoylation in cell- or organism-based systems. To explore a broader extent of chemical space for potential hGOAT inhibitors with improved bioavailability, a fluorescence-based in vitro hGOAT activity assay was used to screen compounds from the Diversity Set IV library (Developmental Therapeutics Program, NCI/NIH) for inhibition activity against GOAT. This library consists of ˜1600 compounds chosen to represent the molecular diversity of “drug-like” molecules within the DTP repository. Compounds were initially screened at 10 and 100 μM, with those compounds exhibiting a dose-dependent decrease in activity and <50% activity at 100 μM verified by a secondary screen under the same conditions. For compounds passing both screens, structurally related compounds were then obtained and assayed from the DTP repository for inhibitory activity against hGOAT.

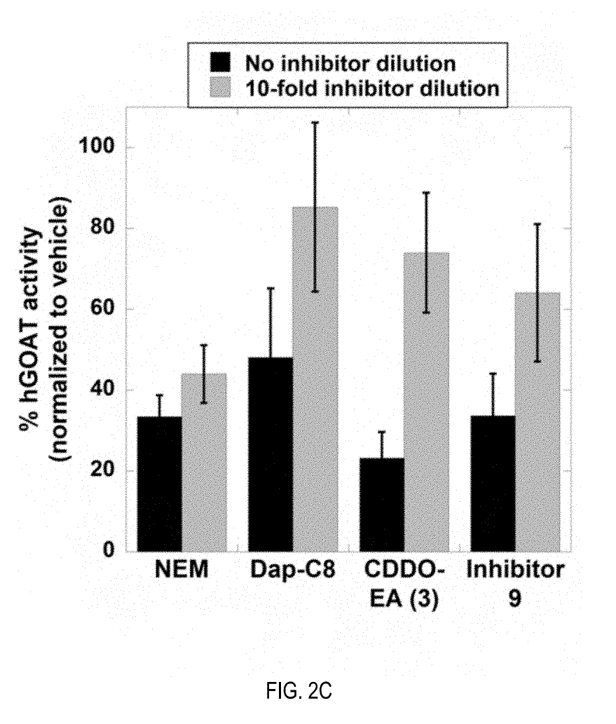

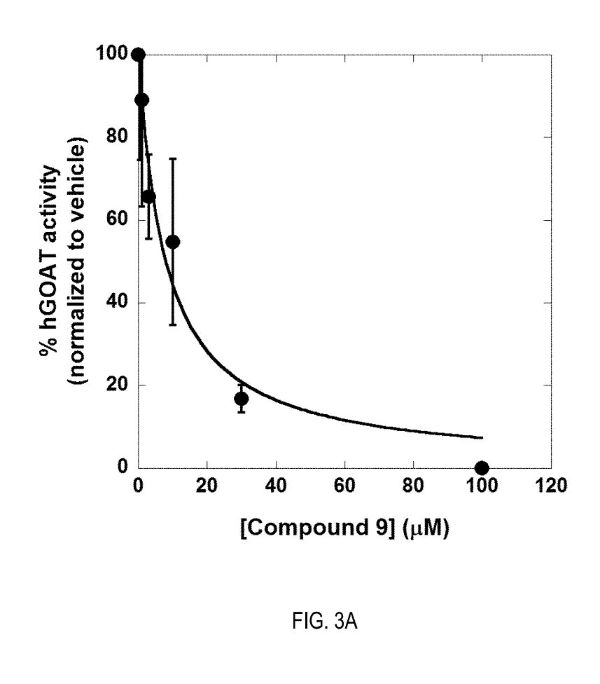

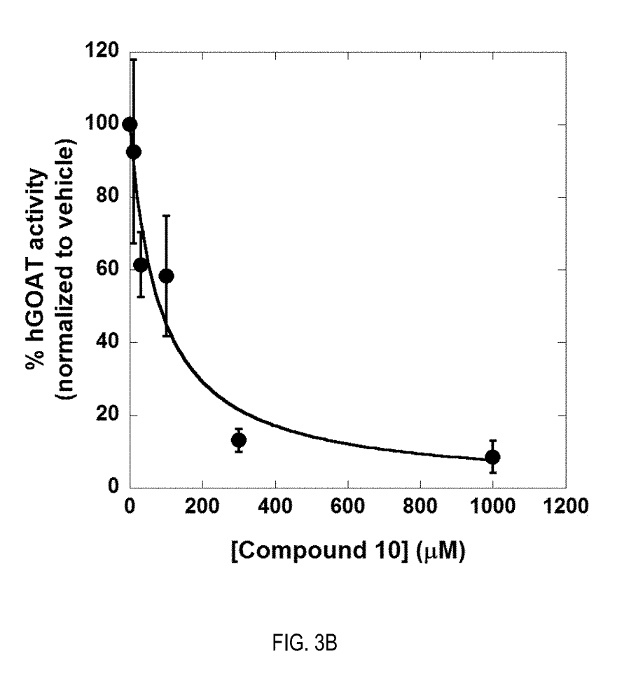

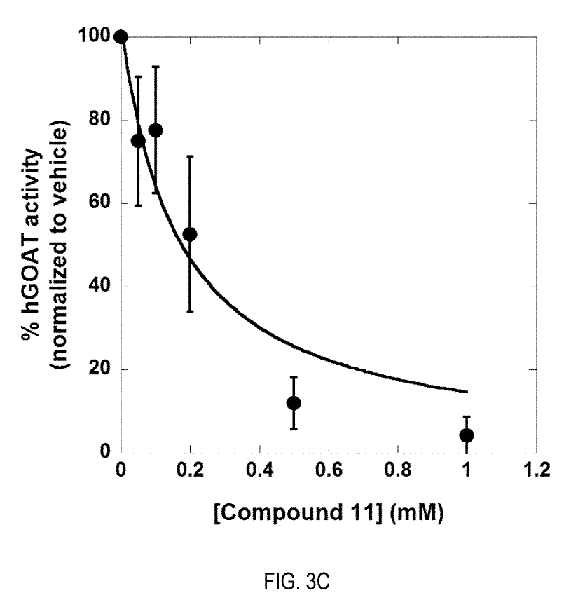

Following this screening protocol, the most promising candidate molecule was identified from the Diversity IV library as a synthetic oleanate triterpenoid, 1[2-cyano-3,12-dioxooleana-1,9-dien-28-oyl]imidazole (CDDO-Im, 1). CDDO-Im inhibits hGOAT activity with an IC₅₀ of 38±6 μM and a structurally related molecule methyl 2-cyano-3,12-dioxooleana-1,9(11)dien-28-oate (CDDO-Me, 2) also exhibits inhibitory activity against hGOAT (FIG. 3).

Verification of CDDO Scaffold Activity Against hGOAT

CDDO-Im and CDDO-Me belong to a class of orally available semisynthetic triterpenoids based on oleanolic acid. This class of compounds has demonstrated antiangiogenic and antitumor activities in animal cancer models through the modulation of multiple signaling pathways including the Nrf2 and NF-κB pathways. Given the inhibition of hGOAT exhibited by CDDO-Im and CDDO-Me, we determined the inhibitory activity of three other CDDO compounds with various carboxyl substituents (compounds 3-5) against hGOAT using the in vitro hGOAT activity assay. Of these five CDDO compounds, all but the acid 5 served as inhibitors against hGOAT-catalyzed ghrelin octanoylation with the ethyl amide derivative (CDDO-EA, 3) demonstrating the most potent inhibition against hGOAT with an IC₅₀ of 8±4 μM. The lack of inhibition activity exhibited by the parent CDDO molecule bearing a carboxylate may reflect a general intolerance for negatively charged groups within the hGOAT active site and substrate binding sites. Substrate selectivity studies of hGOAT have revealed hGOAT does not accept peptide substrates bearing negatively charged side chains or C-terminal acids.

Structure-Activity Analysis of the CDDO Scaffold

These CDDO-derived compounds contain several chemical groups that could be responsible for activity against hGOAT: triterpenoid scaffold; α-cyanoenone (ring A); α,β-unsaturated ketone (ring C). Given the multiple potential pharmacophores within CDDO family compounds and the lack of knowledge regarding the structure and chemical nature of the hGOAT active site and substrate binding sites, the structure-activity parameters defining CDDO-based inhibitor potency against hGOAT were determined. The natural product triterpenoid compounds ursolic acid (6) and oleanic acid (7, from which CDDO is derived) exhibit negligible inhibition of hGOAT activity at concentrations up to 100 μM. These compounds lack the activated α-cyanoenone group shown to be essential for CDDO compound activity in previous studies targeting receptor signaling. However, as both molecules also bear unsubstituted carboxylate groups their lack of hGOAT inhibition could reflect the inability of hGOAT to bind negatively charged molecules. To separate these factors, the ability of the triterpenoid taraxerol (8) to inhibit hGOAT was determined. Taraxerol shares the same scaffold and 3-hydroxyl group as ursolic and oleanic acid but lacks the carboxylic acid. Taraxerol also fails to inhibit hGOAT acylation activity at concentrations up to 100 which suggests hGOAT inhibition by CDDO is not primarily due to the triterpenoid scaffold structure.

Based on the proposed mode of action for CDDO derivatives binding to their receptor targets through modification of reactive cysteine residues, we hypothesized the α-cyanoenone moiety present in the A ring of CDDO derivatives is required for hGOAT inhibition. This group has been shown to covalently modify nucleophilic thiols in a range of protein targets. To examine the effect of a Michael acceptor group on hGOAT inhibition, a series of minimally functionalized steroid derivatives featuring an α,β-unsaturated ketone in the A ring position analogous to that in CDDO-EA (compounds 9-11) was synthesized. All three of these molecules are able to inhibit hGOAT activity, with inhibitor potency scaling with the level of activation of the enone towards nucleophilic addition from the most activated α-cyanoenone 9 (IC₅₀=8±2 μM) to the non-activated enone 11 (IC₅₀=170±60 The α-cyanoenone 9 inhibits hGOAT with potency nearly identical to CDDO-EA 3, indicating the complete triterpenoid scaffold and associate functional groups in the CDDO derivatives are not essential for binding and inhibition of hGOAT.

Through investigation of steroid derivatives, inhibition of hGOAT by steroid derivatives exhibits both chemo- and regioselectivity. Removal of the ketone α,β-unsaturation leads to loss of inhibition by compound 12, with the presence of an α-bromo substituent as a potential electrophile also unable to support inhibition as shown by compound 13. Migration of the α,β-unsaturation to the other side of ring A (compound 14) similarly abrogates inhibition of hGOAT, with the additional alkyl substituent not expected to impact binding to hGOAT based on the tolerance for the larger triterpenoid scaffold in inhibitors 1-4. Furthermore, the lack of hGOAT inhibition exhibited by estrone (15) indicates a planar A ring within a steroid scaffold is insufficient for binding to hGOAT. These findings indicate hGOAT inhibition requires the presence of a specifically located conjugate acceptor group, which is consistent with modification of an enzyme-bound nucleophile within a defined binding pocket on hGOAT.

The equivalent potency of CDDO-EA (3) and α-cyanoenone 9 indicate the distal E ring and carboxyl substituent of CDDO-EA are not required for binding to hGOAT. The contribution of the steroid scaffold to hGOAT binding was determined by measuring inhibition by cyclohexenone and cyclohexenone derivatives (compounds 16-18) which mimic the A ring substitutions of compounds 9-11. Both α-cyanocyclohexenone 16 (1.2±0.2 mM) and α-bromocyclohexenone 17 (IC₅₀=500±100) inhibit hGOAT less potently than their steroid analogues 9 and 10, respectively, while cyclohexenone 18 does not inhibit hGOAT activity at concentrations up to 1 mM. Therefore, the steroid scaffold contributes substantially to inhibitor potency against hGOAT as demonstrated by the ˜150-fold enhancement in context of α-cyanoenones 9 and 16. This enhancement potentially arises from a combination of both increased inhibitor association with hGOAT (better binding) and a decrease in inhibitor reactivity with other microsomal protein targets (reduced competition) due to increased steric congestion from the quaternary center adjacent to the electrophilic β-carbon in α-cyanoenone 9.

Inhibitor Structure Function Analysis Supports a Functionally Essential Cysteine in hGOAT

The requirement for an α,β-unsaturated ketone in hGOAT inhibitors and the increased activity of the triterpenoid and steroid α-cyanoenone compounds suggests these compounds could block hGOAT activity through alkylation of a nucleophilic cysteine residue involved in hGOAT catalysis. The ability of cysteine alkylation to inactivate hGOAT was established by enzyme incubation with N-ethylmaleimide (NEM), a common thiol-modifying reagent. NEM efficiently inhibits hGOAT with a similar IC₅₀ value to those observed for the activated cyclohexenone derivatives 16 and 17, consistent with the involvement of a functionally essential cysteine residue in ghrelin octanoylation by hGOAT.

Previous studies of α-cyanoenone compounds demonstrate these molecules act as covalent reversible inhibitors, with a retro-Michael elimination facilitated by the increased acidity of the α-hydrogen geminal to the cyano group. The reversibility of hGOAT inhibition by the α-cyanoenone compounds 3 and 9 was determined by enzyme pretreatment with each inhibitor at three times the measured IC₅₀ concentration, followed by a 10-fold dilution into either reaction buffer or buffer containing the same inhibitor concentration as the pretreatment. NEM exhibits classical irreversible hGOAT inhibition, with no increase in hGOAT activity following inhibitor dilution. An established GOAT inhibitor, GS(octanamide-Dap)FL, serves as an control for reversible inhibition as expected for a product mimetic non-covalent inhibitor. Both CDDO-EA (3) and α-cyanoenone 9 display reversible hGOAT inhibition, consistent with previously reported reversibility of CDDO compounds. Taken together, the susceptibility of hGOAT to treatment with NEM and the observed pattern of reversible and irreversible hGOAT inhibition by these Michael acceptors support the requirement for one or more cysteine residues to participate in hGOAT catalysis of ghrelin octanoylation.

Expression and Enrichment of hGOAT.

hGOAT was expressed and enriched in insect (Sf9) cell membrane fractions using a previously published procedure.

Peptide Substrate Fluorescent Labeling.

Peptide substrates were labeled with acrylodan on a cysteine thiol and HPLC purified as previously reported.

hGOAT Activity Assays and Analysis.

hGOAT activity assays were performed using a modification of previously reported protocols. For each assay, membrane fraction from Sf9 cells expressing hGOAT was thawed on ice and passed through an 18-gauge needle 10 times to homogenize. Assays were performed with ˜100 μg of membrane protein, as determined by Bradford assay. Membrane fraction was preincubated with 1 μM methoxy arachadonyl fluorophosphonate (MAFP) and inhibitor or vehicle as indicated in 50 mM HEPES pH 7.0 for 30 minutes at room temperature. Reactions were initiated with the addition of 500 □M octanoyl CoA and 1.5 μM fluorescently-labeled ghrelin mimetic, GSSFLC_{Acrylodan}, for a total volume of 50 μL, and were incubated for 3 hours at room temperature in the dark. Reactions were stopped with the addition of 50 μL of 20% acetic acid in isopropanol, and solutions were clarified by protein precipitation with 16.7 μL of 20% trichloroacetic acid, followed by centrifugation (1,000×g, 1 minute). The supernatant was then analyzed by reverse phase HPLC, as previously described. Data reported are the average of three independent determinations, with error bars representing one standard deviation.

Determination of IC50 Values in In Vitro hGOAT Activity Assay

For inhibition experiments, reactions were performed and analyzed as described in the presence of either inhibitor or vehicle (DMSO or ethanol) as appropriate. The percent activity at each inhibitor concentration calculated from HPLC data using equations 1 and 2:

$$\begin{matrix}\% activity = \frac{\% peptide octanoylation in presence of inhibitor}{\% peptide octanoylation in absence of inhibitor} & ( 1 ) \\ \% peptide octanoylation = \frac{Fluorescence of octanoylated peptide}{\begin{matrix}Total peptide fluorescence \\ ( octanoylated and non \text{-} octanoylated )\end{matrix}} & ( 2 )\end{matrix}$$

To determine the IC₅₀ value, the plot of % activity versus [inhibitor] was fit to equation 3, with % activity₀ denoting hGOAT activity in the presence of the vehicle alone:

$$\% activity = \% {activity}_{0} * ( 1 - \frac{[ inhibitor ]}{[ inhibitor ] + {IC}_{50}} ) \qquad ( 3 )$$

Inhibitor Reversibility Assay

Undiluted homogenized membrane protein fraction containing hGOAT (protein concentration ˜7 μg/μL) was incubated with 10 μM MAFP and 3×IC₅₀ of inhibitor or equal volume vehicle (DMSO or ethanol) for 30 minutes at room temperature. Following preincubation, the membrane fraction-inhibitor solution was diluted 10-fold into a reaction mixture containing 500 μM octanoyl CoA, 1.5 μM GSSFLC_{AcDan}, 50 mM HEPES pH 7.0, and either vehicle or inhibitor (final concentration 3×[IC₅₀]) in a total reaction volume of 50 μL. Reactions were incubated for 3 hours at room temperature in the dark and then analyzed as described above.

### Synthetic Methods and Characterization

### (5 S,8S,9S,10S,13S,14S)-10,13-dimethyl-3-oxo-4,5,6,7,8,9,10,11,12,13,14,15,16,17-tetradecahydro-3H-cyclopenta[a]phenanthrene-2-bromide (10)

To a solution of cyclohexenone 11 (0.100 g, 0.367 mmol) in DCM (1 mL) under argon, bromine (0.019 mL, 0.367 mmol) dissolved in DCM (1 ml) was added dropwise over 15 min at 0. Triethyl amine (0.087 mL, 0.623 mmol) was added and the resulting mixture was allowed to warm to rt and stirred for another 1.5 h before it was quenched with 1MHCl. The layers separated and organic layer was washed twice with sodium thiosulphate, then combined, dried over sodium sulfate, and concentrated under reduced pressure. The concentrated reaction mixture was purified with flash column chromatography using 2% ethyl acetate/98% hexane as the eluent which afforded 10 (0.103 g, 80%) as a white solid.

10. mp=111-116; TLC R_{f}=0.52 (10% ethyl acetate/90% hexanes); IR 3.3 (CH₂Cl₂ film) 2964, 2848, 1691, 1436, 954, 755 cm⁻¹; [α]_{D}^{23.3}=+22.3 (c 0.5, CHCl₃); ¹H NMR 400 MHz, CDCl₃) δ 7.60 (s, 1H), 2.56-2.43 (m, 2H), 2.04-1.96 (m, 1H), 1.83-1.57 (m, 6H), 1.48-1.34 (m, 5H), 1.22-1.12 (m, 3H), 1.05 (s, 3H), 1.02-0.93 (m, 3H), 0.73 (s, 3H); ¹³C NMR (75 MHz, CDCl₃) δ 191.5, 158.9, 123.1, 54.4, 50.2, 44.2, 42.8, 41.0, 40.9, 40.2, 38.5, 35.7, 31.5, 27.2, 25.4, 21.4, 20.5, 17.6, 12.9. Anal. Calcd for C₁₉H₂₇BrO: C, 64.96; H, 7.75. Found: C, 64.72; H, 7.94.

### (5S,8S,9S,10S,13S,14S)-10,13-dimethyl-3-oxo-4,5,6,7,8,9,10,11,12,13,14,15,16,17-tetradecahydro-3H-cyclopenta[a]phenanthrene-2-carbonitrile (9)

Procedure based on a similar transformation reported by Fu and Gribble. (Fu, L.; Gribble, G. W. Efficient and Scalable Synthesis of Bardoxolone Methyl (CDDO-methyl Ester). Org. Lett. 2013, 15, 1622-1625).

To a stirred solution of bromo enone (0.210 g, 0.597 mmol) in anhydrous dimethyl formamide (5.9 mL) was added copper (I) cyanide (0.059 g, 0.657 mmol) and potassium iodide ((0.02 g, 0.2 mmol), and the resulting reaction mixture was heated to 120° C. for 36 h. After the completion of reaction, it was cooled to room temperature, quenched with water (5 mL), and diluted with ethyl acetate (15 mL). The organic phase was washed with saturated NaHCO₃ (2×5 mL), brine (5 mL), and dried over Na₂SO₄. Removal of solvent and flash column chromatography over silica gel using 8% ethyl acetate/92% hexanes provided 0.07 g (40%) of 9 as white solid. mp=171-173° C.; TLC R_{f}=0.61 (20% ethyl acetate/80% hexanes); IR (CH₂Cl₂, film) 3411, 2230, 1693, 1447, 1216 cm⁻¹; [α]_{D}^{26.2}=+20.1 (c 0.05, DCM); ¹H NMR 400 MHz, CDCl₃) δ 7.87 (s, 1H), 2.47-2.34 (m, 2H), 2.00-1.92 (m, 1H), 1.84-1.54 (m, 7H), 1.50-1.38 (m, 5H), 1.22-1.12 (m, 3H), 1.08 (s, 3H), 1.05-0.94 (m, 2H), 0.73 (s, 3H); ¹³C NMR (75 MHz, CDCl3) δ 192.4, 169.8, 115.8, 114.5, 54.3, 49.4, 43.2, 40.9, 40.7, 40.1, 40.1, 38.4, 35.9, 35.8, 31.3, 27.3, 25.3, 22.6, 21.2, 20.5, 17.6, 12.6. Anal. Calcd for C₂₀H₂₇NO: C, 80.76; H, 9.15; N, 4.71. Found: C, 80.08; H, 9.05; N, 4.58.

### (5S,8S,9S,10S,13S,14S)-10,13-dimethyltetradecahydro-1H-cyclopenta[a]phenanthren-3(2H)-one (12)

A solution of 5α-Androstan-3β-ol (1.74 g, 6.29 mmol) in DCM (25 mL) was added to a suspension of pyridinium chlorochromate (2.73 g, 12.6 mmol) and silica gel (2.73 g) in DCM (10 mL). The resulting black orange solution was stirred continuously at rt for 2 h. The reaction mixture was filtered through a plug of silica gel eluting with DCM. The filtrate was concentrated under reduced pressure and purified with silica gel column chromatography (1% ethylacetate 99% hexanes) afforded ketone 12 (1.66 g, 96%) as white solid.

12. ¹H NMR (300 MHz, CDCl₃) δ 2.45-2.22 (m, 3H), 2.11-1.99 (m, 2H), 1.68-1.51 (m, 7H), 1.47-1.28 (m, 6H), 1.23-1.08 (m, 3H), 1.01 (s, 3H), 0.99-0.86 (m, 2H), 0.80-0.74 (m, 1H), 0.72 (s, 3H).

### (2R,5S,8S,9S,10S,13S,14S)-2-bromo-10,13-dimethyltetradecahydro-1H-cyclopenta[a]phenanthren-3(2H)-one (13)

A solution of ketone 12 (0.18 g, 0.66 mmol) in acetic acid (6.60 mL) was warmed to 50° C. Pyridinium tribromide (0.23 g, 0.66 mmol) was added in one portion and the solution was stirred continuously. After several seconds, a precipitate was formed. The precipitate was filtered, dried and further purified using silica gel column chromatography in 1% ethylacetate/99% hexanes to afford bromide 13 (0.14 g, 60%) as white powdery solid.

13. ¹H NMR (300 MHz, CDCl₃) δ 4.73 (dd, J=13.5, 6.5 Hz, 1H), 2.63 (dd, J=13.0, 6.5 Hz, 1H), 2.41-2.38 (m, 2H), 1.84-1.69 (m, 7H), 1.45-1.25 (m, 6H), 1.17-1.11 (m, 3H), 1.07 (s, 3H), 0.99-0.75 (m, 3H), 0.70 (s, 3H).

### (2R,5S,8S,9S,10S,13S,14S)-10,13-dimethyl-4,5,6,7,8,9,10,11,12,13,14,15,16,17-tetradecahydro-3H-cyclopenta[a]phenanthren-3-one (11)

A suspension of bromoketone 13 (0.600 g, 1.70 mmol), lithium bromide (0.884 g, 10.2 mmol), and lithium carbonate (0.752 g, 10.2 mmol) in DMF (6.6 mL) was heated overnight to 80° C. The reaction mixture was cooled to rt before adding it to crushed ice. The quenched reaction mixture was extracted with ethyl acetate (3×20 mL). The organic layers were collected, combined, washed with cold water and brine, dried over sodium sulfate, and concentrated under reduced pressure. The concentrated reaction mixture was purified with flash column chromatography using 2% ethyl acetate/98% hexane as the eluent which afforded 11 (0.37 g, 80%) as a white solid. TLC Rf=0.52 (hexane:ethyl acetate, 9:1); ¹H NMR (400 MHz, CDCl₃) δ 7.13 (d, J=10.2 Hz, 1H), 5.82 (dd, J=10.2, 0.9 Hz, 1H), 2.34 (dd, J=13.23, 10.59 Hz, 1H), 2.21 (ddd, J=17.6, 4.1, 0.9 Hz, 1H), 1.93-1.85 (m, 1H), 1.79-1.69 (m, 3H), 1.66-1.51 (m, 3H), 1.49-1.33 (m, 5H), 1.18-1.10 (m, 3H), 1.02-0.90 (m, 6H), 0.71 (s, 3H).

### (8S,9S,10R,13R,14S,17R)-10,13-dimethyl-17-((R)-6-methylheptan-2-yl)-6,7,8,9,10,11,12,13,14,15,16,17-dodecahydro-1H-cyclopenta[a]phenanthren-3(2H)-one (14)

14. ¹H NMR (400 MHz, CDCl₃) δ 5.71 (s, 1H), 2.46-2.33 (m, 2H), 2.31-2.22 (m, 1H), 2.05-1.08 (m, 2H), 1.88-1.79 (m, 2H), 1.70 (dd, 13.8, 5.0 Hz, 1H), 1.65-1.21 (m, 10H), 1.17 (s, 3H), 1.15-0.93 (m, 10H), 0.90 (d, 6.5 Hz, 3H), 0.85 (dd, J=6.6, 1.8 Hz, 6H), 0.70 (s, 3H).

### 6-oxocyclohex-1-enecarbonitrile (16)

6-oxocyclohex-1-enecarbonitrile (16) was synthesizes using the protocol by Fleming and Shook. ¹H NMR (400 MHz, CDCl₃) δ 7.74 (t, J=4.24 Hz, 1H), 2.60-2.54 (m, 4H), 2.46-2.42 (m, 2H) 2.10 (quintet, J=6.39 Hz, 2H)

### 2-bromocyclohex-2-enone (17)

To a solution of cyclohexenone (1.00 g, 10.4 mmol) in DCM (27 mL) under argon, bromine (0.107 mL, 2.08 mmol) dissolved in DCM (27 ml) was added dropwise over 15 min at 0° C. Triethyl amine (0.497 mL, 3.54 mmol) was then added and the resulting mixture was allowed to warm to rt and stirred for another 15 min before it was quenched with 1M HCl. The layers separated and organic layer was washed twice with sodium thiosulphate, then combined, dried over sodium sulfate, and concentrated under reduced pressure. The concentrated reaction mixture was purified with flash column chromatography using 4% ethyl acetate/96% hexane as the eluent which afforded 17 (1.48 g, 81%) as a white solid.

17. TLC R_{f}=0.53 (hexane:ethyl acetate, 9:1); ¹H NMR (400 MHz, CDCl₃) δ 7.41 (t, J=4.4 Hz, 1H), 2.64-2.60 (m, 2H), 2.46-2.42 (m, 2H) 2.06 (quintet, J=6.43 Hz, 2H).

The compound, (5S,8S,9S,10S,13S,14S)-10,13-dimethyl-3-oxo-4,5,6,7,8,9,10,11,12,13,14,15,16,17-tetradecahydro-3H-cyclopenta[a]phenanthrene-2-carbonitrile, 7, was synthesized as follows. To a stirred solution of bromo enone 6 (0.210 g, 0.597 mmol) in anhydrous dimethylformamide (5.9 mL) was added copper (I) cyanide (0.059 g, 0.657 mmol) and potassium iodide ((0.02 g, 0.2 mmol). The resulting reaction mixture was heated to 120° C. for 36 h. After the completion of reaction, it was cooled to room temperature, quenched with water (5 mL), and diluted with ethyl acetate (15 mL). The organic phase was washed with saturated NaHCO₃ (2×5 mL), brine (5 mL), and dried over Na₂SO₄. Removal of solvent and flash column chromatography with silica gel using 30% ethyl acetate/70% hexanes provided 0.070 g (70%) of the α-cyano enone 7 as white solid. 7. mp=171-173° C.; TLC Rf=0.61 (20% ethyl acetate/80% hexanes); IR (CH2Cl2, film) 3411, 2230, 1693, 1447, 1216 cm-1; [α]_{D}^{26.2}=+20.1 (c 0.05, DCM); 1H NMR (400 MHz, CDCl3) δ 7.87 (s, 1H), 2.47-2.34 (m, 2H), 2.00-1.92 (m, 1H), 1.84-1.54 (m, 7H), 1.50-1.38 (m, 5H), 1.22-1.12 (m, 3H), 1.08 (s, 3H), 1.05-0.94 (m, 2H), 0.73 (s, 3H); 13C NMR (75 MHz, CDCl3) δ 192.4, 169.8, 115.8, 114.5, 54.3, 49.4, 43.2, 40.9, 40.7, 40.1, 40.1, 38.4, 35.9, 35.8, 31.3, 27.3, 25.3, 22.6, 21.2, 20.5, 17.6, 12.6. Anal. Calcd for C₂₀H₂₇NO: C, 80.76; H, 9.15; N, 4.71. Found: C, 80.08; H, 9.05; N, 4.58. Table 1 below addresses exemplary steroid enone derivatives according to the present invention and their efficacy in inhibiting hGOAT activity.

TABLE 1

| | | | IC₅₀ | IC₅₀ |
|---|---|---|---|---|
| | | | (individual trials) | (averaged) |
| Compound | Structure | MW | (μM) | (μM) |
| NSM-4-42 | 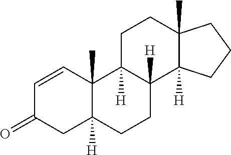 | 272.43 | 201 ± 107 | 166 ± 58 |
| NSM-4-43 | 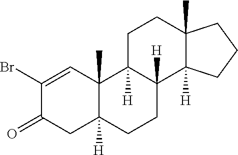 | 351.33 | 84 ± 35 | 83 ± 29 |
| NSM-4-48 | 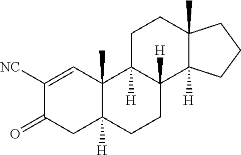 | 297.44 | 7 ± 3 | 8 ± 2 |
| 9 | 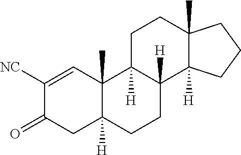 | 297.44 | 7 ± 3 | 8 ± 2 |
| 10 | 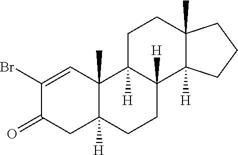 | 351.33 | 84 ± 35 | 83 ± 29 |
| 11 | 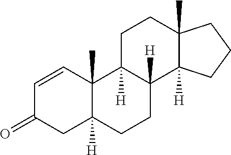 | 272.43 | 201 ± 107 | 166 ± 58 |

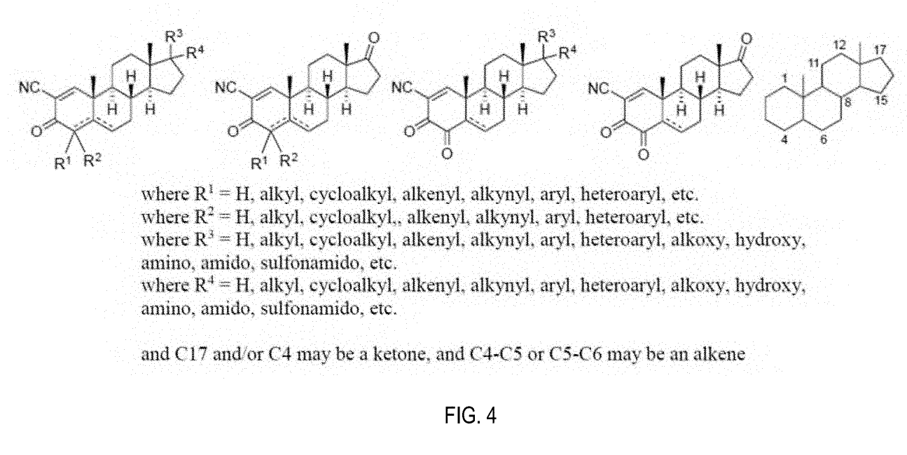

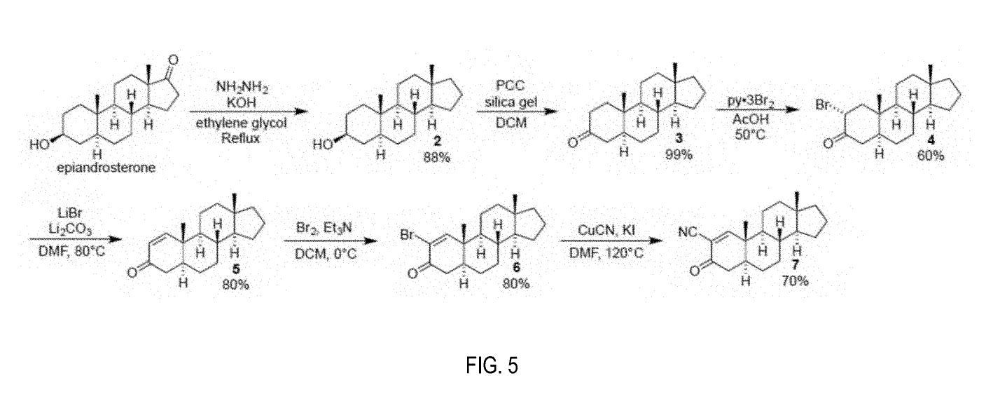

There is seen in FIG. 4, the class of GOAT inhibitors synthesized from epiandrosterone according to the present invention and, in FIG. 5, a method of synthesizing the compounds. The present invention thus encompasses the compounds shown in FIG. 4, wherein R¹ is selected from the group consisting of hydrogen, an alkyl, a cycloalkyl, an alkenyl, and aryl, and a heretoaryl, wherein R² is selected from the group consisting of hydrogen, an alkyl, a cycloalkyl, an alkenyl, and aryl, and a heretoaryl, wherein R³ is selected from the group consisting of hydrogen, an alkyl, a cycloalkyl, an alkenyl, and aryl, a heretoaryl, an alkoxy, a hydroxy, an amino, an amido, and a sulfonamido, and wherein R⁴ is selected from the group consisting of hydrogen, an alkyl, a cycloalkyl, an alkenyl, and aryl, a heretoaryl, an alkoxy, a hydroxy, an amino, an amido, and a sulfonamido Identifying potent GOAT inhibitors is an essential step toward validation and exploitation of the ghrelin-GOAT system for therapeutic targeting. The discovery of this new class of small molecule hGOAT inhibitors will accelerate inhibitor development targeting ghrelin octanoylation, potentially leading to therapeutics for treating diabetes, obesity, and other health conditions impacted by ghrelin signaling.

What is claimed is:

1. A human ghrelin O-acyltransferase inhibitor, comprising a steroid scaffold having α,β-unsaturated ketone in an A ring position.

2. The inhibitor of claim 1 where the α,β-unsaturated ketone comprises an α-cyanoenone.

3. The inhibitor of claim 2 having the formula 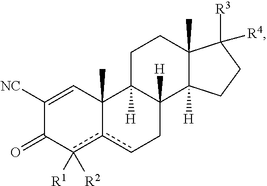
wherein R¹ is selected from the group consisting of hydrogen, an alkyl, a cycloalkyl, an alkenyl, and aryl, and a heretoaryl, wherein R² is selected from the group consisting of hydrogen, an alkyl, a cycloalkyl, an alkenyl, and aryl, and a heretoaryl, wherein R³ is selected from the group consisting of hydrogen, an alkyl, a cycloalkyl, an alkenyl, and aryl, a heretoaryl, an alkoxy, a hydroxy, an amino, an amido, and a sulfonamido, and wherein R⁴ is selected from the group consisting of hydrogen, an alkyl, a cycloalkyl, an alkenyl, and aryl, a heretoaryl, an alkoxy, a hydroxy, an amino, an amido, and a sulfonamido.

4. The inhibitor of claim 2 having the formula 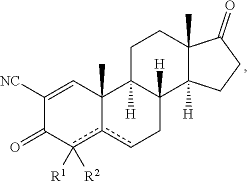
wherein R¹ is selected from the group consisting of hydrogen, an alkyl, a cycloalkyl, an alkenyl, and aryl, and a heretoaryl, wherein R² is selected from the group consisting of hydrogen, an alkyl, a cycloalkyl, an alkenyl, and aryl, and a heretoaryl.

5. The inhibitor of claim 2 having the formula 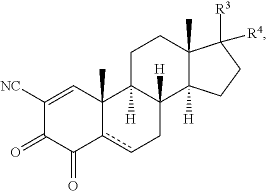
wherein R³ is selected from the group consisting of hydrogen, an alkyl, a cycloalkyl, an alkenyl, and aryl, a heretoaryl, an alkoxy, a hydroxy, an amino, an amido, and a sulfonamido, and wherein R⁴ is selected from the group consisting of hydrogen, an alkyl, a cycloalkyl, an alkenyl, and aryl, a heretoaryl, an alkoxy, a hydroxy, an amino, an amido, and a sulfonamido.

6. The inhibitor of claim 2 having the formula

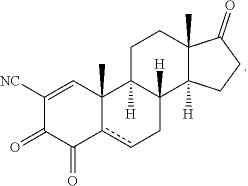

7. A method of inhibiting human ghrelin O-acyltransferase, comprise the step of administering a compound comprising a steroid scaffold having α,β-unsaturated ketone in an A ring position.

8. The method of claim 7 where the α,β-unsaturated ketone comprises an α-cyanoenone.

9. The method of claim 8, wherein the compound has the formula 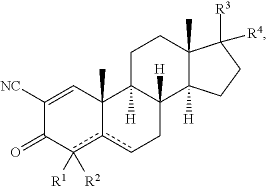
wherein R¹ is selected from the group consisting of hydrogen, an alkyl, a cycloalkyl, an alkenyl, and aryl, and a heretoaryl, wherein R² is selected from the group consisting of hydrogen, an alkyl, a cycloalkyl, an alkenyl, and aryl, and a heretoaryl, wherein R³ is selected from the group consisting of hydrogen, an alkyl, a cycloalkyl, an alkenyl, and aryl, a heretoaryl, an alkoxy, a hydroxy, an amino, an amido, and a sulfonamido, and wherein R⁴ is selected from the group consisting of hydrogen, an alkyl, a cycloalkyl, an alkenyl, and aryl, a heretoaryl, an alkoxy, a hydroxy, an amino, an amido, and a sulfonamido.

10. The method of claim 8, wherein the compound has the formula 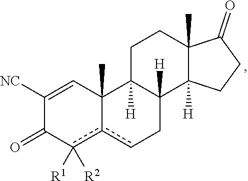
wherein R¹ is selected from the group consisting of hydrogen, an alkyl, a cycloalkyl, an alkenyl, and aryl, and a heretoaryl, wherein R² is selected from the group consisting of hydrogen, an alkyl, a cycloalkyl, an alkenyl, and aryl, and a heretoaryl.

11. The method of claim 8, wherein the compound has the formula 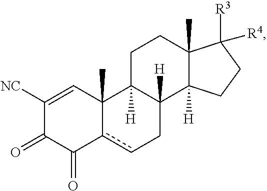
wherein R³ is selected from the group consisting of hydrogen, an alkyl, a cycloalkyl, an alkenyl, and aryl, a heretoaryl, an alkoxy, a hydroxy, an amino, an amido, and a sulfonamido, and wherein R⁴ is selected from the group consisting of hydrogen, an alkyl, a cycloalkyl, an alkenyl, and aryl, a heretoaryl, an alkoxy, a hydroxy, an amino, an amido, and a sulfonamido.

12. The method of claim 8, wherein the compound has the formula [chemical structure]
